(19) United States
(12) Patent Application Publication
(10) Pub. No.: US 2003/0153551 A1
(43) Pub. Date: Aug. 14, 2003

(54) Method for treating ocular neovascular diseases

(76) Inventor: Romulus Kimbro Brazzell, Alpharetta, GA (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/364,607

(22) Filed: Feb. 11, 2003

Related U.S. Application Data
(60) Provisional application No. 60/356,792, filed on Feb. 13, 2002.

(51) Int. Cl. A61K 31/553

(52) U.S. Cl. 514/211.08

(57) ABSTRACT

The invention provides a method for causing regression of ocular neovascularization in a subject by administering an effective amount of a staurosporine derivative to the subject.

The invention relates to the treatment of ocular neovascular diseases by administering certain staurosporine derivatives to subjects in need of treatment.

The invention is directed to methods for the treatment of an ocular neovascular disease comprising administering to a subject suffering from ocular neovascularization an effective amount of a compound of formula I to cause regression of neovascularization in the eye of the subject, wherein formula I is

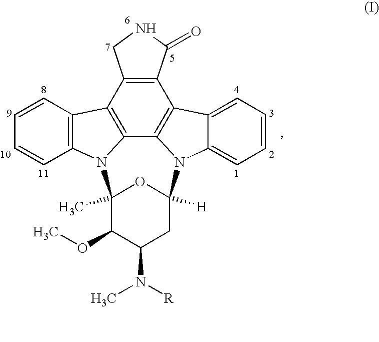

wherein R represents a hydrocarbyl radical R^{o} or an acyl radical Ac.

Suitable hydrocarbyl radicals include acyclic, carbocyclic and carbocyclic-acyclic hydrocarbyl radicals having a maximum total number of carbon atoms of preferably 30, especially 18. Additionally suitable hydrocarbyl radicals are heterocyclic radicals and heterocyclic-acyclic radicals. The hydrocarbyl radicals (R^{O}) may be saturated or unsaturated and substituted or unsubstituted. Suitable acyl radicals include optionally functionally modified carboxylic acid and organic sulfonic acid, and optionally esterified phosphoric acid, e.g., pyro- or ortho-phosphoric acid.

Preferred acyclic hydrocarbyl radicals include C₁-C₂₀-alkyl radicals; C₂-C₂₀ hydroxyalkyl radicals of which the hydroxy group is in any position other than the 1-position; cyano-[C₁-C₂₀]-alkyl radicals; carboxy-[C₁-C₂₀]-alkyl radicals of which the carboxy group; and C₃-C₂₀-alkenyl radicals of which the free valency is not at the same carbon atom as the double bond. Exemplary acyclic hydrocarbyl radicals are radicals of lower alkyl, e.g., methyl, ethyl, propyl, butyl, pentyl, hexyl and heptyl; lower alkenyl, e.g., propenyl, 2- or 3-methallyl and 2- or 3-butenyl; lower alkadienyl, e.g., 1-penta-2,4-dinyl; and lower alkynyl, e.g., propargyl or 2-butynyl. Preferred carbocyclic hydrocarbyl radicals are radicals of mono-, bi- or polycyclic cycloalkyl, e.g., cyclopropyl, cyclobutyl, cyclopentyl, cyclohexyl, bicyclo[2,2,2]octyl, 2-bycyclo[2,2,1]heptyl and adamantyl; cycloalkenyl; cycloalkandienyl; and corresponding aryl. Aryl radicals include radicals of phenyl, napthyl (e.g., 1- or 2-napthyl), biphenylyl (e.g., 4-biphenylyl), anthryl, fluorenyl, azulenyl, and aromatic analogues thereof having one or more saturated rings. Preferred carbocyclic-acyclic radicals are acyclic radicals that carry one or more of carbocyclic radicals. Heterocyclic radicals and heterocyclic-acyclic radicals include monocyclic, bicyclic, polycyclic, aza-, thia-, oxa-, thaza-, oxaza-, diaza-, triaza-, and tetraza-cyclic radicals of aromatic character.

Exemplary acyl radicals derived from an optionally functionally modified carboxylic acid (Ac¹) have the formula Z-C(═W)—in which W is oxygen, sulfur, or imino and Z is hydrogen, hydrocarbyl R^{O}, hydrocarbyloxy R^{O}O, or amino. Preferably, W is oxygen or sulfur, and Z is C₁-C₇ alkyl, especially C₁-C₄ alkyl, which is optionally substituted by halogen, carboxy or C¹-C₄ alkoxycarbonyl. Additionally preferred Z is phenyl, pyridyl, furyl, thienyl, imidazolyl, quinolyl, isoquinolyl, benzofuranyl or benzimidazolyl, each of which is unsubstituted or substituted by C₁-C₄ alkyl, C₁-C₄ alkoxy, halogen, nitro, trifluoromethyl, carboxy, C₁-C₄ alkoxycarbonyl, methylenedioxy, cyano and/or a salt thereof. Preferred Ac¹ acyl radicals have the formula R_{b}^{O}—CO—, in which R_{b}^{O} is hydrogen, benzoyl, or a hydrocarbyl radical, e.g., C₁-C₁₉ alkyl radical which is optionally substituted by a carboxy group, cyano group, ester group, amino group or helogen. Another group of preferred Ac¹ acyl radicals have the formula R_{O}—O—CO—. Yet another group of preferred Ac¹ acyl radicals have the formula

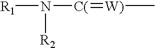

in which R¹ and R₂ are independently selected from hydrogen and unsubstituted acyclic C₁-C₇ hydrocarbyl, preferably C₁-C₄ alkyl and C₃-C₇ alkenyl. R₁ and R₂, independently, can be monocyclic aryl, aralkyl or aralkenyl having a maximum of 10 carbon atoms, each of which is optionally substituted by C₁-C₄ alkyl, C₁-C₄ alkoxy, halogen and/or nitro. Particularly desirable radicals of this group have hydrogen as R₁ and optionally substituted C₁-C₄ alkyl, C₃-C₇ alkenyl, phenyl, pyridyl, pyrimidyl, furyl, thienyl, imidazolyl, quinolyl, isoquinolyl, benzofuranyl or benzimidazolyl as R₂.

Exemplary acyl radicals derived from an organic sulfonic acid (Ac2) have the formula R^{O}—SO₂— in which R^{O} is a hydrocarbyl radical. Preferably, R^{O} of the sulfonic acid acyl radicals is C₁-C₇ alkyl, phenyl, pyridyl, pyrimidyl, furyl, thienyl, imidazolyl, quinolyl, isoquinolyl, benzofuranyl or benzimidazolyl, each of which is unsubstituted or is substituted by C₁-C₄ alkyl, C₁-C₄ alkoxy, halogen, nitro, trifluoromethyl, carboxy, C₁-C₄ alkoxycarbonyl, methylenedioxy, cyano and/or a salt thereof.

Exemplary acyl radicals derived from optionally esterified phosphoric acid (Ac³) have the formula

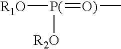

in which R₁ and R₂ are independently selected from hydrogen, unsubstituted acyclic C₁-C₇ hydrocarbyl, preferably C₁-C₄ alkyl and C₃-C₇ alkenyl. R₁ and R₂, independently, can be monocyclic aryl, aralkyl or aralkenyl having a maximum of 10 carbon atoms, each of which is optionally substituted by C₁-C₄ alkyl, C₁-C₄ alkoxy, halogen and/or nitro.

Additionally suitable staurosporine derivatives include staurosporines of formula (I) in which R is derived from an a-amino acid selected from glycine, phenylglycine, alanine, phenylalanine, proline, leucine, serine, valine, tyrosine, arginine, histidine and asparagine, or a salt thereof. Suitable staurosporine derivatives for the present invention are disclosed in further detail in U.S. Pat. No. 5,093,330 to Caravatti et al. The description of the staurosporine derivatives in the patent is herein incorporated by reference.

Particularly preferred staurosporine derivatives of formula (I) for the present invention include:

N-(3-carboxypropionyl)-staurosporine, N-benzoyl-staurosporine, N-trifluoracetyl-staurosporine, N-methylaminothiocarbonyl-staurosporine, N-phenylcarbamoyl-staurosporine, N-(3-nitrobenzoyl)-staurosporine, N-(3-fluorobenzoyl)-staurosporine, N-tert-butoxycarbonyl-staurosporine, N-(4-carboxybenzoyl)-staurosporine, N-(3,5-dinitrobenzoyl)-staurosporine, N-alanyl-staurosporine, N-ethyl-staurosporine, N-carboxymethyl-staurosporine, N-[(tert.-butoxycarbonylamino)-acetyl]-staurosporine, N-(2-aminoacetyl)-staurosporine, and pharmaceutically acceptable salts thereof.

The pharmaceutical composition of the present invention which contains a compound of formula I as the active ingredient can be administered enterally, nasally, buccally, rectally, topically, orally, and parenterally, e.g., intravenous, intramuscular, intravitreal, subconjunctival or subcutaneous administration, to treat ocular neovascularization in mammalian subjects, especially human. The compositions may contain the active ingredient alone or, preferably, the active ingredient along with a pharmaceutically acceptable carrier. The effective dosage of the active ingredient depends on the type of targeted disease, as well as the species, age, weight and physical condition of the subject, pharmacokinetic data, and the mode of administration.

To “to cause regression of neovascularization” as used herein means to decrease the amount of neovasculature in the eye of a subject afflicted with an ocular neovascular disease. Ocular neovascular disease, as used herein, includes, e.g., exudative age related macular degeneration, proliferative diabetic retinopathy and ischemic retinopathy.

The compounds of formula I is administered in an amount effective to cause regression of neovacularization in the eye of a subject mammal, e.g., a human. For an individual having a bodyweight of about 70 kg, the daily systemic dose administered is from about 0.1 g to about 20 g, preferably from about 0.5 g to about 5 g, of the active ingredient, and suitable pharmaceutical compositions may have from about 1% to about 95% of the active ingredient. Suitable unit dose forms include coated and uncoated tablets, ampoules, vials, suppositories, or capsules. Other suitable dosage forms include injectables, intraocular devices, intravitreal devices, ointments, creams, pastes, foams, tinctures, lip-sticks, eye-drops, oral-drops, sprays, dispersions and the like. The pharmaceutical compositions of the present invention are prepared in a manner known in the art, for example, by means of conventional mixing, granulating, coating, dissolving or lyophilizing processes.

Preference is given to the use of solutions of the active ingredient, and also suspensions or dispersions, especially isotonic aqueous solutions, dispersions or suspensions. Suitable pharmaceutical compositions containing the active ingredient may have carriers, e.g., mannitol and starch, preservatives, stabilizers, wetting agents, emulsifiers, solubilizers, salts for regulating osmotic pressure, buffers and the like. The compositions are prepared in a manner known in the art, for example by means of conventional dissolving and lyophilizing processes. A solution or suspension form of the composition may contain viscosity-increasing agents, e.g., sodium carboxymethylcellulose, carboxymethylcellulose, dextran, polyvinylpyrrolidone, and gelatins; and solubilizers, e.g., Tween 80 [polyoxyethylene(20)sorbitan mono-oleate; trademark of ICI Americas, Inc, USA].

Suitable carriers include fillers, e.g., sugars, for example lactose, saccharose, mannitol or sorbitol; cellulose preparations; calcium phosphates, e.g., tricalcium phosphate and calcium hydrogen phosphate; binders, e.g., starches, methylcellulose, hydroxypropyl methylcellulose, sodium carboxymethylcellulose and polyvinylpyrrolidone; and, if desired, disintegrators, e.g., starches, crosslinked polyvinylpyrrolidone, alginic acid or salts thereof. Additional suitable excipients are flow conditioners and lubricants, e.g., silicic acid, talc, stearic acid and salts thereof, such as magnesium or calcium stearate, polyethylene glycol, and derivatives thereof.

When administered to a subject in need of treatment, the active ingredient, i.e., a staurosporine derivative, of the present invention causes regression of ocular neovascularization, especially retinal neovascularization and chorodial neovascularization, without any identifiable toxic effect on mature retinal vessels.

The present invention is further illustrated with the following examples. However, these are not to be construed as limiting the invention thereto.

### EXAMPLE 1

Ischemic retinopathy is produced in C57/BL6J mice by a method described by Smith et al., Oxygen-induced Retinopathyin the Mouse, Invest. Ophthalmol. Vis. Sci. 35, 101-111 (1994). Seven-day-old mice and their mothers are placed in an airtight incubator and exposed to an atmosphere of 75+3% oxygen for 5 days. Incubator temperature is maintained at 23±2° C., and oxygen is measured every 8 hours with an oxygen analyzer. After 5 days, the mice are removed from the incubator, placed in room air, and subjected to a drug treatment. N-benzoyl-staurosporine (NBS) is dissolved in dimethyl sulfoxide (DMSO) and diluted to final concentrations with water; the maximum concentration of DMSO is 1%. Vehicle (1% DMSO) or vehicle containing various concentrations of the drug (volume=10 μl per gram body weight) is placed in the stomach by gavage. Different mice are given 60, 300 or 600 mg of NBS per kg of body weight. As a control, a group of mice are given the vehicle without NBS.

After 5 days of treatment, the mice are sacrificed, eyes are rapidly removed and frozen in optimum cutting temperature embedding compound (OCT; Miles Diagnostics, Elkhart, Ind.) or fixed in 10% phosphate-buffered formalin and embedded in paraffin. Adult C57BL6J mice are also treated by gavage with the drug or vehicle and after 5 days, they are sacrificed and their eyes are processed for frozen or paraffin sections.

Frozen sections (10 μm) of the eyes from drug-treated and control mice are histochemically stained with biotinylated griffonia simplicifolia lectin B4 (Vector Laboratories, Burlingame, Calif.) which selectively binds to endothelial cells. Slides are incubated in methanol/H₂O₂ for 10 minutes at 4° C., washed with 0.05 M Tris-buffered saline, pH 7.6 (TBS), and incubated for 30 minutes in 10% normal porcine serum. Slides are incubated 2 hours at room temperature with biotinylated lectin and after rinsing with 0.05M TBS, they are incubated with avidin coupled to peroxidase (Vector Laboratories) for 45 minutes at room temperature. After being washed for 10 minutes with 0.05 M TBS, slides are incubated with diaminobenzidine to give a brown reaction product. Some slides are counterstained with hematoxyln and all were mounted with Cytoseal.

To perform quantitative assessments, 10 μm serial sections are cut through half of each eye and sections roughly 50-60 μm apart are stained with lectin, providing 13 sections per eye for analysis. Lectin-stained sections are examined with an Axioskop microscope (Zeiss, Thornwood, N.Y.) and images are digitized using a 3 CCD color video camera (IK-TU40A, Toshiba, Tokyo, Japan) and a frame grabber. Image-Pro Plus software (Media Cybernetics, Silver Spring, Md.) is used to delineate lectin-stained cells on the surface of the retina and their area is measured. The mean of the 13 measurements from each eye is used as a single experimental value.

The mice with ischemic retinopathy treated with the vehicle without NBS show a marked increase in the area of endothelial cell staining throughout the retina with large clumps of cells on the retinal surface when compared to nonischemic mice, which show normal vessels in the superficial and deep capillary beds with a few connecting vessels. Ischemic mice that are given 600 mg/kg of NBS once a day for 5 days have a dramatic decrease in endothelial cell staining on the surface and within the retina when compared to the vehicle-treated mice. In fact, the endothelial cell staining within the retina of the NBS-treated ischemic mice is less than that of the nonischemic mice. High magnification shows that there are no identifiable endothelial cells on the surface of the retina, indicating that there is complete inhibition of neovascularization. There is also a striking absence of endothelial cell staining in the inner nuclear layer and outer plexiform layer where the deep capillary beds are normally located.

The mice with ischemic retinopathy which are given 300 mg/kg or 60 mg/kg of NBS twice a day by gavage show some clumps of neovascularization on the surface of the retina that is less than clumps in the retina of the vehicle-treated control mice. The retinas of NBS-treated mice also show some decrease in endothelial staining within the retina. The result of the image analysis demonstrated that the endothelial cell staining on and in the retinas of mice treated with 600 or 60 mg/kg once a day, was significantly less than that in vehicle-treated mice and showed a dose-dependent effect when compared to the mice that were treated twice a day. The results clearly demonstrate that the staurosporine derivative inhibits retinal neovascularization.

### EXAMPLE 2

Adult C57BL6J mice are given the vehicle or 600 mg/kg of NBS by gavage once a day and after 5 days, they are sacrificed and their eyes are processed as in Example 1.

Image analysis shows that there is no difference in the total area of endothelial staining in the retina or the appearance of retinal vessels in the NBS-treated mice compared to vehicle-treated mice. The analysis also shows no difference in the amount of retinal endothelial cell staining between the NBS- and vehicle-treated mice. This demonstrates that the staurosporine derivative is not toxic to endothelial cells of mature vessels.

### EXAMPLE 3

Litters of newborn C57/BL6J mice (neonatal mice) are divided into treatment and control groups which received daily subcutaneous injections of 100 mg/kg of the drug or vehicle, respectively. At 7 or 10 days of age, the mice are anesthetized with ether, and perfused with 1 ml of phosphate-buffered saline containing 50 mg/ml of fluorescein-labeled dextran (2×10⁶ average mw, Sigma, St. Louis, Mo.) as described by Tobe et al., Evolution of Neovascularization in Mice with Overexpression of VEGF in Photoreceptors, Invest. Ophthalmol. Vis. Sci. 39, 180-188 (1998). The eyes are removed and fixed for 1 hour in 10% phosphate-buffered formalin. The cornea and lens are removed and the entire retina is carefully dissected from the eyecup. Radially cuts are made from the edge of the retina to the equator in all 4 quadrants, and the retina is flat-mounted in Aquamount with photoreceptors facing upward. The flat mounts are examined by fluorescence microscopy, and the images are digitized using a 3 CCD. color video camera and a frame grabber. Image-Pro™ Plus is used to measure the distance from the center of the optic nerve to the leading front of developing retinal vessels in each quadrant and the mean is used as a single experimental value.

At 7 days of age, retinal vessels in the vehicle-treated mice almost reach the peripheral edge of retina, but in the NBS-treated mice, retinal vessels only extend slightly more than halfway to the periphery. At 10 days of age, the superficial capillary bed is complete and extends all the way to the peripheral edge of the retina, and the deep capillary bed is partially developed. But in the NBS-treated mice, the superficial capillary bed has not yet reached the edge of the retina. The distance from the optic nerve to the vascular front is calculated by image analysis and the differences between the treated and control mice at 7 and 10 days of age is statistically significant. This indicates that the staurosporine derivative inhibits retinal vascular development.

### EXAMPLE 4

C57BL/6J mice are treated in accordance with the Association for Research in Vision and Ophthalmology resolution for the treatment of animals. Choroidal neovascularization is generated by modification of a previously described technique, Tobe et al. Targeted disruption of the FGF2 gene does not prevent choroidal neovascularization in a murine model, Amer. J Path, in press. Briefly, 4 to 5 week old male C57BL/6J mice are anesthetized with ketamine hydrochloride (100 mg/kg body weight) and the pupils are dilated with 1% tropicamide. Three burns of krypton laser photocoagulation (100 μm spot size, 0.1 seconds duration, 150 mW) are delivered to each retina using the slit lamp delivery system of a Coherent Model 920 Photocoagulator and a hand held cover slide as a contact lens. Burns are performed in the 9, 12, and 3 o'clock positions of the posterior pole of the retina. Production of a bubble at the time of laser, which indicates rupture of Bruch's membrane, is an important factor in obtaining CNV, so only mice in which a bubble is produced for all three burns are included. Ten mice are randomly assigned to treatment with vehicle alone, and ten mice are assigned to receive vehicle containing 400 mg/kg/day of NBS given orally by gavage. After 14 days, the mice are killed with an overdose of pentobarbital sodium, and their eyes are rapidly removed and frozen in optimal cutting temperature embedding compound (OCT). Frozen serial sections (10 μm) are cut through the entire extent of each burn and histochemically stained with biotinylated griffonia simplicifolia lectin B4 (Vector Laboratories, Burlingame, Calif.), which selectively binds to endothelial cells. Slides are incubated in methanol/H₂O₂ for 30 minutes at 4° C., washed with 0.05 M Tris-buffered saline, pH 7.4 (TBS), and incubated for 30 minutes in 10% normal swine serum. Slides are rinsed with 0.05M TBS and incubated 2 hours at 37° C. with biotinylated lectin. After being rinsed with 0.05M TBS, slides are incubated with Streptavidin-phosphatase (Kirkegaard and Perry Laboratories, Cabin John, Md.) for 30 minutes at room temperature. After a 10 minute incubation in 0.05 M Tris buffer, pH 7.6, slides are developed in Histomark Red (Kirkegaard and Perry) to give a red reaction product, and mounted with Cytoseal (Stephens Scientific, Riverdale, N.J.). Some slides are counterstained with Contrast Blue (Kirkegaard and Perry).

To perform quantitative assessments, lectin-stained sections are examined with an Axioskop microscope (Zeiss, Thornwood, N.Y.) and images are digitized using a 3 CCD color video camera (IK-TU40A, Toshiba, Tokyo, Japan) and a frame grabber. Image-Pro Plus software (Media Cybernetics, Silver Spring, Md.) is used to delineate and measure the area of lectin-stained blood vessels in the subretinal space. For each lesion, area measurements are made for all sections on which some of the lesion appeared and added together to give the integrated area measurement. Values are averaged to give one experimental value per mouse. A 2-sample t-test for unequal variances is performed to compare the log mean integrated area between treatment and control mice.

Two weeks after laser, all lesions in both groups of mice show a discontinuity in Bruch's membrane with roughly equivalent damage to the overlying retina. All mice treated with vehicle alone show large areas of choroidal neovascularization at the site of each laser-induced rupture of Bruch's membrane. There is proliferation of retinal pigmented epithelial cells along the margin of the new vessels. Retinal blood vessels stained with lectin are seen in the overlying retina. In contrast, all mice given 400 mg/kg/day of NBS have very little if any choroidal neovascularization at the site of each laser-induced rupture of Bruch's membrane. In most instances, there is no identifiable lectin-stained neovascular tissue throughout the entire burn, but some burns contained regions in which there are thin discs of lectin-stained tissue. There is mild proliferation of RPE cells. Despite the marked decrease in choroidal neovascularization in the eyes of treated mice, the overlying retinal vessels appear normal. This is best seen in sections with no counterstain.

Quantitation of the integrated area of lectin staining per lesion shows a dramatic decrease in the mice treated with NBS (0.0090182±0.0017540 mm²) compared to lesions in mice treated with vehicle alone (0.0695621±0.0073960 mm²). This difference is highly statistically significant (p=0.004). These results clearly demonstrate that the staurosporine derivative dramatically inhibits chorodial neovascularization.

1. A method for the treatment of an ocular neovascular disease comprising administering to a subject suffering from ocular neovascularization an effective amount of a compound of formula I to cause regression of neovascularization in the eye of the subject, wherein formula I is 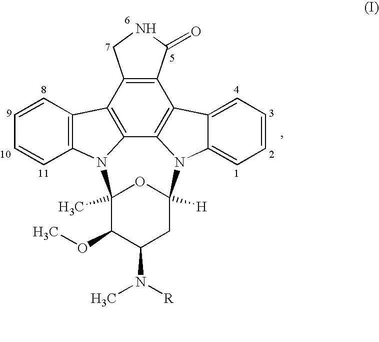
wherein R represents a hydrocarbyl radical R^{O} or an acyl radical Ac, or a salt thereof.

2. The method of claim 1 wherein said hydrocarbyl radical is an acyclic, carbocyclic, carbocyclic-acyclic, heterocyclic or heterocyclic-acyclic hydrocarbyl radical.

3. The method of claim 2 wherein said acyclic hydrocarbyl radical is a radical of a C₁-C₂₀-alkyl radical, C₂-C₂₀ hydroxyalkyl radical of which the hydroxy group is in any position other than the 1-position, cyano-[C₁-C₂₀1-alkyl radical, carboxy-[C₁-C₂₀]-alkyl radical of which the carboxy group, or C₃-C₂₀-alkenyl radical of which the free valency is not at the same carbon atom as the double bond.

4. The method of claim 2 wherein said carbocyclic hydrocarbyl radical is a radical of mono-, bi- or polycyclic cycloalkyl; cycloalkenyl; cycloalkandienyl; and aryl.

5. The method of claim 2 wherein said carbocyclic-acyclic radicals is an acyclic radical that carry one or more of carbocyclic radicals, and said heterocyclic radical and heterocyclic-acyclic radical are monocyclic, bicyclic, polycyclic, aza-, thia-, oxa-, thaza-, oxaza-, diaza-, triaza-, and tetraza-cyclic radicals of aromatic character.

6. The method of claim 1 wherein said acyl radical is an optionally functionally modified carboxylic acid, organic sulfonic acid, or optionally esterified phosphoric acid.

7. The method of claim 6 wherein said acyl radical has the formula Z-C(═W )—, wherein W is oxygen, sulfur, or imino and Z is hydrogen, C₁-C₇ alkyl, amino, phenyl, pyridyl, furyl, thienyl, imidazolyl, quinolyl, isoquinolyl, benzofuranyl or benzimidazolyl.

8. The method of claim 6 wherein said acyl radical has the formula R_{b}^{O}—CO—, wherein R_{b}^{O} is hydrogen, benzoyl, or a C₁-C₁₉ alkyl radical.

9. The method of claim 6 wherein said acyl radical has the formula R^{O}—O—CO—, wherein R^{O} is an acyclic, carbocyclic, carbocyclic-acyclic, heterocyclic or heterocyclic-acyclic hydrocarbyl radical.

10. The method of claim 6 wherein said acyl radical has the formula 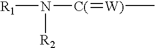
wherein R₁ and R₂ are independently selected from hydrogen and unsubstituted acyclic C₁-C₇ hydrocarbyl.

11. The method of claim 6 wherein said acyl radical has the formula R^{O}—SO₂— wherein R^{O} is a hydrocarbyl radical.

12. The method of claim 6 wherein said acyl radical has the formula 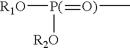
in which R₁ and R₂ are independently selected from hydrogen, unsubstituted acyclic C₁-C₇ hydrocarbyl.

13. The method of claim 1 wherein said active ingredient is selected from N-(3-carboxypropionyl)-staurosporine, N-benzoyl-staurosporine, N-trifluoracetyl-staurosporine, N-methylaminothiocarbonyl-staurosporine, N-phenylcarbamoyl-staurosporine, N-(3-nitrobenzoyl)-staurosporine, N-(3-fluorobenzoyl)-staurosporine, N-tert-butoxycarbonyl-staurosporine, N-(4-carboxy benzoyl )-staurosporine, N-(3,5-dinitrobenzoyl)-staurosporine, N-alanyl-staurosporine, N-ethyl-staurosporine, N-carboxymethyl-staurosporine, N-[(tert-butoxycarbonylamino)-acetyl]-staurosporine, N-(2-aminoacetyl)-staurosporine, and salts thereof.

14. The method of claim 1 wherein said active ingredient is N-benzoyl-staurosporine or a salt thereof.

15. The method of claim 14, wherein said ocular neovascular disease is selected from the group consisting of choroidal neovascularization and retinal neovascularization.

16. The method of claim 14, wherein said ocular neovascular disease is exudative age related macular degeneration.

17. The method of claim 14, wherein said ocular neovascular disease is proliferative diabetic retinopathy.

18. The method of claim 14, wherein said ocular neovascular disease is an ischemic retinopathy.
